(19) United States
(12) Patent Application Publication
(10) Pub. No.: US 2011/0306598 A1
(43) Pub. Date: Dec. 15, 2011

(54) Use of the association of a sinus node if current inhibitor and an angiotensin-converting enzyme inhibitor in the treatment of heart failure

(75) Inventors: Christian Thuillez, Rouen (FR); Paulus Mulder, Mont Saint Aignan (FR); Jean-Paul Vilaine, Chatenay Malabry (FR); Marie-Dominique Fratacci, Bois D'Arcy (FR); Guy Lerebours-Pigeonniere, Levallois Perret (FR); Luc Feldmann, Paris (FR); Jérôme Roussel, Viroflay (FR)

(73) Assignee: LES LABORATOIRES SERVIER, SURESNES CEDEX (FR)

(21) Appl. No.: 13/134,667

(22) Filed: Jun. 14, 2011

(30) Foreign Application Priority Data
Jun. 15, 2010 (FR) 1002525

(51) Int. Cl. A61K 31/55; A61P 9/00

(52) U.S. Cl. 514/212.07

(57) ABSTRACT

Use of the association of a selective and specific sinus node I_{f} current inhibitor, more especially ivabradine or N-{[(7S)-3,4-dimethoxybicyclo[4.2.0]octa-1,3,5-trien-7-yl]methyl}-3-(7,8-dimethoxy-1,2,4,5-tetrahydro-3H-3-benzazepin-3-yl)-N-methyl-3-oxo-1-propanamine, and an agent that inhibits angiotensin-converting enzyme in obtaining medicaments intended for the treatment of heart failure, more especially heart failure having preserved systolic function.

Medicinal products containing the same which are useful in the treatment of heart failure.

The present invention relates to the use of the association of a selective and specific sinus node I_{f} current inhibitor and an agent that inhibits angiotensin-converting enzyme (ACE inhibitor) in obtaining medicaments intended for the treatment of heart failure, more especially heart failure with preserved systolic function.

The present invention relates more especially to the use of the association of a selective and specific sinus node I_{f} current inhibitor and an agent that inhibits angiotensin-converting enzyme wherein the selective and specific sinus node I_{f} current inhibitor is selected from:
ivabradine, or 3-{3-[{[(7S)-3,4-dimethoxybicyclo[4.2.0]octa-1,3,5-trien-7-yl]-methyl}(methyl)amino]propyl}-7,8-dimethoxy-1,3,4,5-tetrahydro-2H-3-benzazepin-2-one, of formula (I):

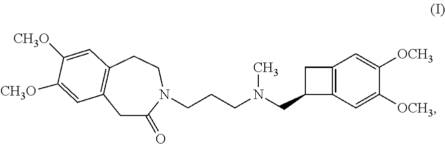

and its addition salts with a pharmaceutically acid, their hydrates and crystalline forms,
N-{[(7S)-3,4-dimethoxybicyclo[4.2.0]octa-1,3,5-trien-7-yl]methyl}-3-(7,8-dimethoxy-1,2,4,5-tetrahydro-3H-3-benzazepin-3-yl)-N-methyl-3-oxo-1-propanamine of formula (II)

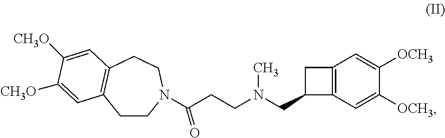

and its addition salts with a pharmaceutically acid, their hydrates and crystalline forms.

Amongst the pharmaceutically acceptable acids there may be mentioned, without implying any limitation, hydrochloric acid, hydrobromic acid, sulphuric acid, phosphoric acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, benzenesulphonic acid, camphoric acid, pamoic acid and 1,5-naphthalenedisulphonic acid.

Selective and specific sinus node I_{f} current inhibitors, and more especially:
ivabradine, and its addition salts with a pharmaceutically acceptable acid, and more especially its hydrochloride, their hydrates and crystalline forms,
N-{[(7S)-3,4-dimethoxybicyclo[4.2.0]octa-1,3,5-trien-7-yl]methyl}-3-(7,8-dimethoxy-1,2,4,5-tetrahydro-3H-3-benzazepin-3-yl)-N-methyl-3-oxo-1-propanamine, and its addition salts with a pharmaceutically acceptable acid, and more especially its hydrochloride and its fumarate, their hydrates and crystalline forms, have very valuable pharmacological and therapeutic properties, especially negative chronotropic (heart-rate-reducing) properties, making those compounds useful in treating or preventing, or improving the prognosis of, various cardiovascular diseases associated with myocardial ischaemia such as angina pectoris, myocardial infarct and associated rhythm disturbances, and also in various pathologies involving rhythm disturbances, especially supraventricular rhythm disturbances, and in chronic heart failure.

The preparation and therapeutic use of ivabradine and its addition salts with a pharmaceutically acceptable acid, more especially its hydrochloride, have been described in European patent specification EP 0 534 859.

The preparation and therapeutic use of N-{[(7S)-3,4-dimethoxybicyclo[4.2.0]octa-1,3,5-trien-7-yl]methyl}-3-(7,8-dimethoxy-1,2,4,5-tetrahydro-3H-3-benzazepin-3-yl)-N-methyl-3-oxo-1-propanamine and its addition salts with a pharmaceutically acceptable acid, more especially its hydrochloride and its fumarate, have been described in European patent specification EP 2 036 892.

Angiotensin-converting enzyme inhibitors are one of the major therapeutic classes in the treatment of arterial hypertension. They act principally by inhibiting the synthesis of angiotensin II and by blocking the breakdown of bradykinin.

In addition to the lowering of arterial pressure, they have been shown to improve the morbidity (myocardial infarct, cerebral vascular accidents) and cardiovascular mortality of hypertensive patients, diabetic patients and patients with pre-existing coronary disease.

The Applicant has discovered that the association of a selective and specific sinus node I_{f} current inhibitor, more especially:
ivabradine, or 3-{3-[{[(7S)-3,4-dimethoxybicyclo[4.2.0]octa-1,3,5-trien-7-yl]-methyl}(methyl)amino]propyl}-7,8-dimethoxy-1,3,4,5-tetrahydro-2H-3-benzazepin-2-one, or
N-{[(7S)-3,4-dimethoxybicyclo[4.2.0]octa-1,3,5-trien-7-yl]methyl}-3-(7,8-dimethoxy-1,2,4,5-tetrahydro-3H-3-benzazepin-3-yl)-N-methyl-3-oxo-1-propanamine,
   and an agent that inhibits angiotensin-converting enzyme has valuable properties allowing its use in the treatment of heart failure, more especially heart failure with preserved systolic function.

Heart failure due to systolic dysfunction of the left ventricle is not the only form of heart failure. Increasingly often, patients with heart failure have an ejection fraction which is greater than 40%. The proportion of heart failure referred to as “diastolic heart failure” (or rather “heart failure with preserved systolic function”) increases with age. It currently accounts for 30 to 40% of hospital admissions for heart failure and, after the age of 80, its frequency exceeds that of heart failures due to systolic dysfunction. Diastolic heart failures generally feature both prolonged ventricular relaxation and a reduction in the distensibility of the left ventricle chamber. The basic causes are ischaemic, hypertensive and elderly-patient cardiopathies. Predisposing factors are age, sex (women), diabetes, obesity and arterial hypertension. Concentric remodelling of the left ventricle, with or without hypertrophy, consistently gives rise to disruption of diastolic function. In most cases a triggering factor is found to be the cause of a congestive attack. The frequency of “diastolic” heart failure increases with age. Its physiopathology remains complex and merits being better understood by clinicians.

No treatment has hitherto demonstrated efficacy in this pathology, the mortality (50% at 4 years) of which corresponds to that of systolic heart failure.

The Applicant has discovered that the use of the association of a selective and specific sinus node I_{f} current inhibitor and an agent that inhibits angiotensin-converting enzyme makes it possible to obtain pharmacological effects that are superior to those observed when using either a selective and specific sinus node I_{f} current inhibitor on its own or an agent that inhibits angiotensin-converting enzyme on its own. The use of the association of a selective and specific sinus node I_{f} current inhibitor and an agent that inhibits angiotensin-converting enzyme moreover makes it possible for the observed physiological parameters to return to values very close to normal. These observations make it possible to envisage using the association of a selective and specific sinus node I_{f} current inhibitor and an agent that inhibits angiotensin-converting enzyme in the treatment of heart failure, more especially heart failure having preserved systolic function.

The selective and specific sinus node I_{f} current inhibitor used will preferably be selected from:
ivabradine, in the form of the hydrochloride or one of its hydrates or crystalline forms, and
N-{[(7S)-3,4-dimethoxybicyclo[4.2.0]octa-1,3,5-trien-7-yl]methyl}-3-(7,8-dimethoxy-1,2,4,5-tetrahydro-3H-3-benzazepin-3-yl)-N-methyl-3-oxo-1-propanamine, in the form of the hydrochloride or fumarate or one of their hydrates or crystalline forms.

The agent that inhibits angiotensin-converting enzyme is preferably selected from the following compounds: perindopril, optionally in the form of its active metabolite perindoprilat, ramipril, optionally in the form of its active metabolite ramiprilat, enalapril, optionally in the form of its active metabolite enalaprilat, captopril, lisinopril, delapril, fosinopril, quinapril, spirapril, imidapril, trandolapril, optionally in the form of its active metabolite trandolaprilat, benazepril, cilazapril, temocapril, alacepril, ceronapril, moveltipril and moexipril, and their addition salts with pharmaceutically acceptable acids or bases, their hydrates and their crystalline forms.

Preferably used agents that inhibit angiotensin-converting enzyme are perindopril, captopril, enalapril, ramipril, lisinopril, benazepril, quinapril and delapril, and their addition salts with pharmaceutically acceptable acids or bases, their hydrates and crystalline forms.

The agent that inhibits angiotensin-converting enzyme which is even more preferably used is perindopril or one of its addition salts with a pharmaceutically acceptable acid or base, and more especially its tert-butylamine or arginine salts, their hydrates and crystalline forms.

The present invention relates also to pharmaceutical compositions comprising as active ingredients:
ivabradine, or one of its hydrates, crystalline forms, and addition salts with a pharmaceutically acceptable acid and more especially its hydrochloride, or N-{[(7S)-3,4-dimethoxybicyclo[4.2.0]octa-1,3,5-trien-7-yl]methyl}-3-(7,8-dimethoxy-1,2,4,5-tetrahydro-3H-3-benzazepin-3-yl)-N-methyl-3-oxo-1-propanamine or one of its addition salts with a pharmaceutically acceptable acid, and more especially its hydrochloride or its fumarate, their hydrates or crystalline forms, and
perindopril, or one of its addition salts with a pharmaceutically acceptable base, and more especially its tert-butylamine or arginine salts, their hydrates or crystalline forms,
   for use in the treatment of heart failure, more especially heart failure with preserved systolic function.

The pharmaceutical compositions that may be used are those that are suitable for oral, parenteral or nasal administration, tablets, dragées, sublingual tablets, capsules, lozenges, suppositories, creams, ointments, dermal gels etc. and also pharmaceutical compositions having programmed, delayed, prolonged or deferred release.

Besides the selective and specific sinus node I_{f} current inhibitor and the compound that inhibits angiotensin-converting enzyme, said pharmaceutical compositions comprise one or more excipients or carriers selected from diluents, lubricants, binders, disintegration agents, absorbents, colourants, sweeteners etc.

By way of non-limiting example there may be mentioned:
as diluents: lactose, dextrose, sucrose, mannitol, sorbitol, cellulose, glycerol,
as lubricants: silica, talc, stearic acid and its magnesium and calcium salts, polyethylene glycol,
as binders: magnesium aluminium silicate, starch, gelatin, tragacanth, methylcellulose, sodium carboxymethylcellulose and polyvinylpyrrolidone,
as disintegrants: agar, alginic acid and its sodium salt, effervescent mixtures.

The useful dosage varies according to the sex, age and weight of the patient, the administration route, the nature of the disorder and of any associated treatments and ranges from 2.5 to 30 mg of ivabradine per 24 hours, and more preferably from 5 to 15 mg per day, and even more preferably from 10 to 15 mg per day. The dose of N-{[(7S)-3,4-dimethoxybicyclo[4.2.0]octa-1,3,5-trien-7-yl]methyl}-3-(7,8-dimethoxy-1,2,4,5-tetrahydro-3H-3-benzazepin-3-yl)-N-methyl-3-oxo-1-propanamine (hereinafter referred to as compound A) may vary from 5 to 100 mg per day. The dose of the agent that inhibits angiotensin-converting enzyme may be less than that used when it is administered on its own.

When the agent that inhibits angiotensin-converting enzyme is perindopril, its daily dose will preferably be between 1 and 10 mg inclusive.

The present invention relates also to the association of N-{[(7S)-3,4-dimethoxybicyclo[4.2.0]octa-1,3,5-trien-7-yl]methyl}-3-(7,8-dimethoxy-1,2,4,5-tetrahydro-3H-3-benzazepin-3-yl)-N-methyl-3-oxo-1-propanamine, or one of its addition salts with a pharmaceutically acceptable acid, their hydrates or crystalline forms, and perindopril, or one of its addition salts with a pharmaceutically acceptable base, and more especially its tert-butylamine or arginine salts, their hydrates or crystalline forms.

The Examples that follow illustrate the invention.

### LIST OF ABBREVIATIONS USED

dP/dtₘₐₓ: maximum increase in pressure per second
dP/dtₘᵢₙ: maximum reduction in pressure per second
HF: heart failure
LVEDP: Left Ventricular End Diastolic Pressure
LVEDPVR: Left Ventricular End Diastolic Pressure Volume Relation
LVESP: Left Ventricular End Systolic Pressure
LVESPVR: Left Ventricular End Systolic Pressure Volume Relation
LV: left ventricle

### Pharmacological Tests:

Heart failure was induced in rats by ligature of the left coronary artery (the control animals undergo an operation but are not ligated), which causes ischaemia of part of the wall of the left ventricle. The animals recuperate for 7 days and then, for 12 weeks, they are given either 3 mg/kg of compound A, or 0.4 mg/kg of perindopril, or perindopril and compound A concomitantly.

Twelve weeks after the operation it is found that the animals having undergone coronary ligature develop heart failure that is both systolic (ejection anomaly) and diastolic (filling anomaly).

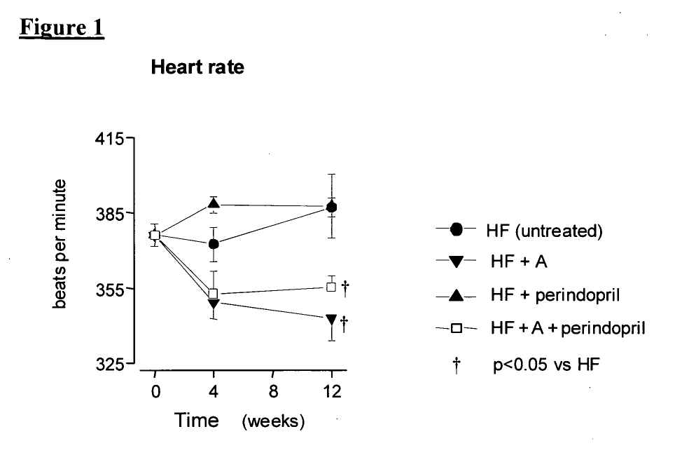

In those animals, compound A, on its own or in association with perindopril, makes possible a significant reduction in heart rate (Table 1 and FIG. 1).

TABLE 1

| | | | HF | | HF + | HF + A + |
|---|---|---|---|---|---|---|
| | | | (untreated) | HF + A | perindopril | perindopril |
| Heart rate | Duration | 4 weeks | 372.5 | 349.3 | 388.2 | 352.8 |
| (bpm) | of treatment | 12 weeks | 387.2 | 342.7^{†} | 387.7 | 353.1^{†} |

^{†}p < 0.05 vs HF

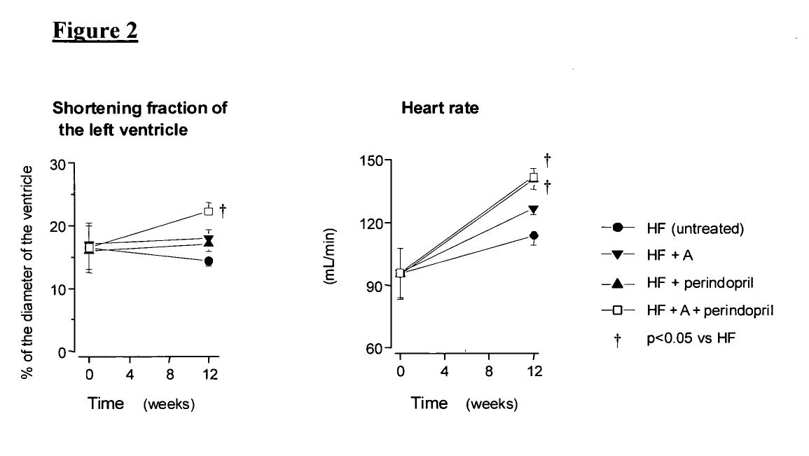

Co-treatment with perindopril and compound A makes possible a significant increase in the shortening fraction of the left ventricle, that is to say an improvement in its contractility (Table 2 and FIG. 2). Consequently, heart rate is improved compared to animals with heart failure that are not given treatment.

TABLE 2

| | HF | | HF + | HF + A + |
|---|---|---|---|---|
| | (untreated) | HF + A | perindopril | perindopril |
| shortening | 14.4 | 18.0 | 17.1 | 22.3^{†} |
| fraction (% of the | | | | |
| diameter of the LV) | | | | |
| heart rate (mL/min) | 114 | 127 | 142^{†} | 140^{†} |

^{†}p < 0.05 vs HF

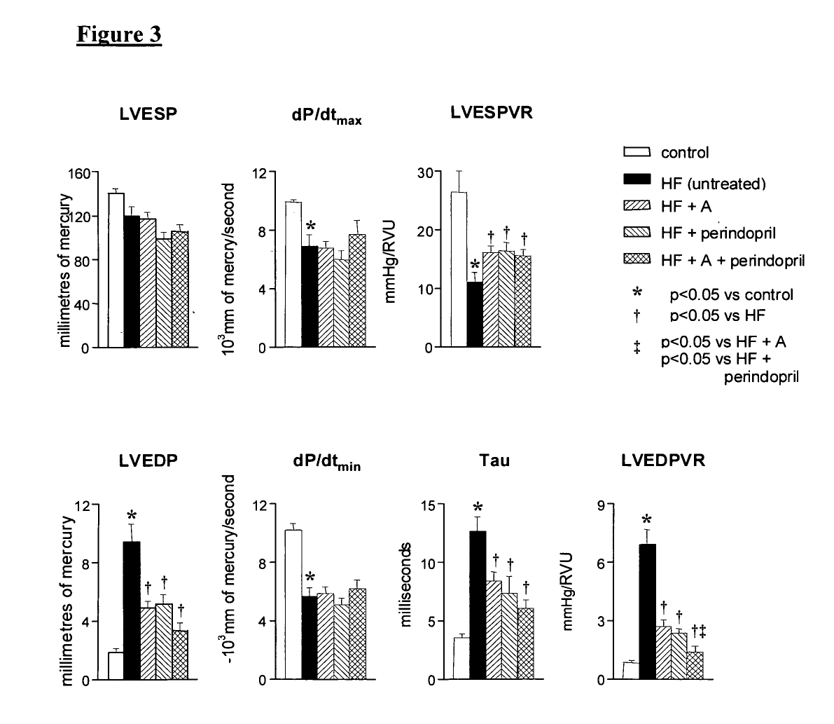

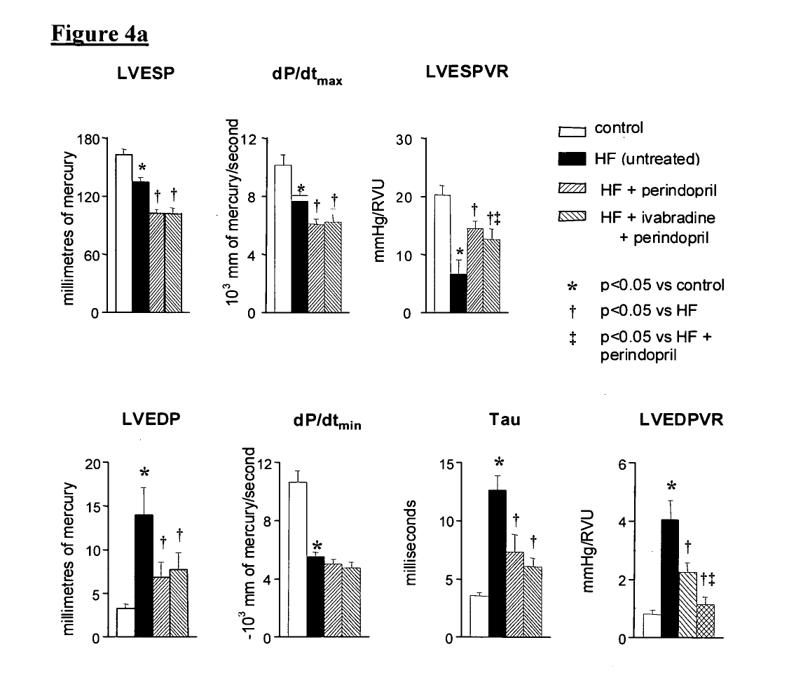

As Table 3 shows (FIG. 3), the various systolic and diastolic parameters are modified by heart failure. The left ventricle contracts less well (dP/dtₘₐₓ and LVESPVR significantly lower in the HF animals than in the healthy controls), which indicates systolic impairment. There is a major deterioration in diastolic function: the pressure inside the ventricle at the end of diastole is raised (LVEDP), the relaxation time (tau) is lengthened and the compliance (ability of the ventricle to distend) is low (LVEDPVR increased).

TABLE 3

| | | HF | | HF + | HF + A + |
|---|---|---|---|---|---|
| | Control | (untreated) | HF + A | perindopril | perindopril |
| LVESP | 140 | 120 | 118 | 99 | 105 |
| (mmHg) | | | | | |
| dP/dtₘₐₓ | 9.92 | 6.89* | 6.78 | 5.97 | 7.69 |
| (10³ | | | | | |
| mmHg/s) | | | | | |
| LVESPVR | 26.4 | 11.1* | 16.1^{†} | 16.4^{†} | 15.6^{†} |
| (mmHg/ | | | | | |
| RVU) | | | | | |
| LVEDP | 1.86 | 9.43* | 4.89^{†} | 5.17^{†} | 3.32^{†} |
| (mmHg) | | | | | |
| dP/dtₘᵢₙ | 10.24 | 5.66* | 5.87 | 5.11 | 6.19 |
| (−10³ | | | | | |
| mmHg/s) | | | | | |
| tau (ms) | 3.54 | 12.64* | 8.37^{†} | 7.31^{†} | 6.05^{†} |
| LVEDPVR | 0.84 | 6.93* | 2.70^{†} | 2.36^{†} | 1.37^{†‡} |
| (mmHg/ | | | | | |
| RVU) | | | | | |

*p < 0.05 vs control;
^{†}p < 0.05 vs HF;
^{‡}p < 0.05 vs HF + A and vs HF + perindopril

It is found that treatment of the animals which have heart failure, whether with perindopril on its own or with compound A on its own, improves systolic function, which can be seen from the LVESPVR, the only load-independent parameter.

The end diastolic pressure and relaxation time are clearly improved by perindopril on its own or by compound A on its own, and a tendency to a further reduction in those two parameters is noted when the two substances are administered together. The compliance of the left ventricle (measured by LVEDPVR), the only load-independent parameter, is very clearly improved by perindopril and by compound A. Surprisingly, this effect is significantly increased when the animals are given the two treatments concomitantly.

In fact, the association of compound A and perindopril makes it possible to significantly improve the compliance, which returns to a level close to that of the control animals. The association of perindopril and N-{[(7S)-3,4-dimethoxybicyclo[4.2.0]octa-1,3,5-trien-7-yl]methyl}-3-(7,8-dimethoxy-1,2,4,5-tetrahydro-3H-3-benzazepin-3-yl)-N-methyl-3-oxo-1-propanamine accordingly makes it possible to improve the deterioration in diastolic function.

This effect on systolic function and diastolic function was then studied using the association of perindopril and a different I_{f} current inhibitor, ivabradine.

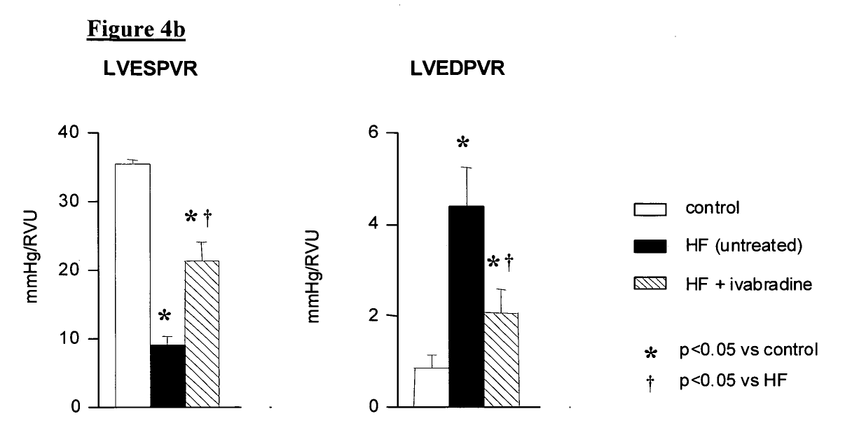

It is found that treatment with perindopril on its own or in association with ivabradine improves the systolic function (Table 4a and FIG. 4a).

With respect to the diastolic dysfunction, treatment with perindopril and ivabradine is clearly more effective than perindopril on its own (the effect of ivabradine on its own is comparable to that of perindopril on its own, cf. Table 4b and FIG. 4b). The compliance of the left ventricle is returned to a level similar to that of the healthy animals.

TABLE 4a

| | | | | HF + |
|---|---|---|---|---|
| | | HF | HF + | ivabradine + |
| | Control | (untreated) | perindopril | perindopril |
| LVESP (mmHg) | 163 | 134* | 102^{†} | 100^{†} |
| dP/dtₘₐₓ (10³ mmHg/s) | 10.11 | 7.68* | 6.08^{†} | 6.10^{†} |
| LVESPVR | 20.2 | 6.6* | 14.5^{†} | 12.6^{†‡} |
| (mmHg/RVU) | | | | |
| LVEDP (mmHg) | 3.29 | 13.93* | 6.88^{†} | 5.01^{†} |
| dP/dtₘᵢₙ | 10.63 | 5.54* | 4.99 | 4.97 |
| (−10³ mmHg/s) | | | | |
| tau (ms) | 3.21 | 14.29* | 10.92^{†} | 8.52^{†} |
| LVEDPVR | 0.79 | 4.06* | 2.25^{†} | 1.15^{†‡} |
| (mmHg/RVU) | | | | |

*p < 0.05 vs control;
^{†}p < 0.05 vs HF;
^{‡}p < 0.05 vs HF + A and vs HF + perindopril

TABLE 4B

| | | HF | HF + |
|---|---|---|---|
| | Control | (untreated) | ivabradine |
| LVESPVR (mmHg/RVU) | 35.53 | 9.66* | 20.63*^{†} |
| LVEDPVR (mmHg/RVU) | 0.85 | 5.33* | 1.87*^{†} |

*p < 0.05 vs control;
^{†}p < 0.05 vs HF

These experiments show that, in a model of heart failure, the association of a selective and specific sinus node I_{f} current inhibitor and an agent that inhibits angiotensin-converting enzyme makes possible an improvement in diastolic function which is greater than that obtained with one of those two treatments used on its own, this improvement allowing a return to normal diastolic function.

### Pharmaceutical Compositions:

Formula for the preparation of 1000 tablets each containing as active ingredients 7.5 mg of ivabradine and 2 mg of perindopril tert-butylamine:

| | | |
|---|---|---|
| Ivabradine hydrochloride | 8.085 | g |
| Perindopril tert-butylamine | 2 | g |
| Lactose monohydrate | 62 | g |
| Magnesium stearate | 1.3 | g |
| Povidone | 9 | g |
| Anhydrous colloidal silica | 0.3 | g |
| Cellulose sodium glycolate | 30 | g |
| Stearic acid | 2.6 | g |

Formula for the preparation of 1000 tablets each containing as active ingredients 10 mg of compound A and 2 mg of perindopril tert-butylamine:

| | | |
|---|---|---|
| Compound A fumarate | 12.48 | g |
| Perindopril tert-butylamine | 2 | g |
| Lactose monohydrate | 62 | g |
| Magnesium stearate | 1.3 | g |
| Povidone | 9 | g |
| Anhydrous colloidal silica | 0.3 | g |
| Cellulose sodium glycolate | 30 | g |
| Stearic acid | 2.6 | g |

Other examples of pharmaceutical compositions according to the invention are given hereinbelow, without implying any limitation:

| | | | Perindopril tert- | Perindopril |
|---|---|---|---|---|
| | Ivabradine | Compound A | butylamine | arginine |
| Example | (mg) | (mg) | salt (mg) | salt (mg) |
| 1 | 10 | — | 2 | — |
| 2 | 15 | — | 4 | — |
| 3 | 10 | — | — | 2.5 |
| 4 | 15 | — | — | 5 |
| 5 | — | 60 | 2 | — |
| 6 | — | 80 | 4 | — |
| 7 | — | 60 | — | 2.5 |
| 8 | | 80 | — | 5 |

1. A method of treating heart failure in a subject in need thereof, comprising administration of an effective amount of a composition comprising a combination of a selective and specific sinus node I_{f} current inhibitor and an agent that inhibits angiotensin-converting enzyme.

2. The method of claim 1, wherein the heart failure is heart failure with preserved systolic function.

3. The method of claim 1, wherein the selective and specific sinus node I_{f} current inhibitor is:
ivabradine, or 3-{3-[{[(7S)-3,4-dimethoxybicyclo[4.2.0]octa-1,3,5-trien-7-yl]-methyl}(methyl)amino]propyl}-7,8-dimethoxy-1,3,4,5-tetrahydro-2H-3-benzazepin-2-one, or one of its addition salts with a pharmaceutically acceptable acid, or hydrates or crystalline forms thereof, or
N-{[(7S)-3,4-dimethoxybicyclo[4.2.0]octa-1,3,5-trien-7-yl]methyl}-3-(7,8-dimethoxy-1,2,4,5-tetrahydro-3H-3-benzazepin-3-yl)-N-methyl-3-oxo-1-propanamine, or one of its addition salts with a pharmaceutically acceptable acid, or hydrates or crystalline forms thereof.

4. The method of claim 1, wherein the selective and specific sinus node I_{f} current inhibitor is ivabradine, in the form of the hydrochloride, or one of its hydrates or crystalline forms.

5. The method of claim 1, wherein the selective and specific sinus node I_{f} current inhibitor is N-{[(7S)-3,4-dimethoxybicyclo[4.2.0]octa-1,3,5-trien-7-yl]methyl}-3-(7,8-dimethoxy-1,2,4,5-tetrahydro-3H-3-benzazepin-3-yl)-N-methyl-3-oxo-1-propanamine, in the form of the hydrochloride or fumarate, or one of its hydrates or crystalline forms.

6. The method of claim 1, wherein the agent that inhibits angiotensin-converting enzyme is perindopril, or one of its addition salts with a pharmaceutically acceptable base, or one of its hydrates or crystalline forms.

7. The method of claim 6, wherein the perindopril is in the form of a tert-butylamine or arginine salt or one of its hydrates or crystalline forms.

8. The method of claim 1, wherein the selective and specific sinus node I_{f} current inhibitor is ivabradine, in the form of the hydrochloride or one of its hydrates or crystalline forms, and the agent that inhibits angiotensin-converting enzyme is perindopril, in the form of the tert-butylamine or arginine salt or one of its hydrates or crystalline forms.

9. The method of claim 1, wherein the selective and specific sinus node I_{f} current inhibitor is N-{[(7S)-3,4-dimethoxybicyclo[4.2.0]octa-1,3,5-trien-7-yl]methyl}-3-(7,8-dimethoxy-1,2,4,5-tetrahydro-3H-3-benzazepin-3-yl)-N-methyl-3-oxo-1-propanamine, in the form of the hydrochloride or fumarate or one of its hydrates or crystalline forms, and the agent that inhibits angiotensin-converting enzyme is perindopril, in the form of the tert-butylamine or arginine salt or one of its hydrates or crystalline forms.

10. A method of treating heart failure in a subject in need thereof, comprising administration of an effective amount of a pharmaceutical composition comprising as active ingredients:
ivabradine, in the form of the hydrochloride or one of its hydrates or crystalline forms, and
perindopril, in the form of the tert-butylamine or arginine salt or one of their hydrates or crystalline forms,
alone or in combination with one or more pharmaceutically acceptable excipients.

11. The method of claim 10, wherein the heart failure is heart failure with preserved systolic function.

12. A pharmaceutical composition comprising as active ingredients:
N-{[(7S)-3,4-dimethoxybicyclo[4.2.0]octa-1,3,5-trien-7-yl]methyl}-3-(7,8-dimethoxy-1,2,4,5-tetrahydro-3H-3-benzazepin-3-yl)-N-methyl-3-oxo-1-propanamine, in the form of the hydrochloride or fumarate or one of its hydrates or crystalline forms, and
perindopril, in the form of the tert-butylamine or arginine salt or one of its hydrates or crystalline forms,
alone or in combination with one or more pharmaceutically acceptable excipients.

13. A method of treating heart failure in a subject in need thereof, comprising administration of an effective amount of the pharmaceutical composition of claim 12.

14. The method of claim 13, wherein the heart failure is heart failure with preserved systolic function.

15. A composition comprising a combination of N-{[(7S)-3,4-dimethoxybicyclo[4.2.0]octa-1,3,5-trien-7-yl]methyl}-3-(7,8-dimethoxy-1,2,4,5-tetrahydro-3H-3-benzazepin-3-yl)-N-methyl-3-oxo-1-propanamine or one of its addition salts with a pharmaceutically acceptable acid, or hydrates or crystalline forms thereof, and perindopril, in the form of the tert-butylamine or arginine salt, or hydrates or crystalline forms thereof.

16. The composition of claim 15, wherein N-{[(7S)-3,4-dimethoxybicyclo[4.2.0]octa-1,3,5-trien-7-yl]methyl}-3-(7,8-dimethoxy-1,2,4,5-tetrahydro-3H-3-benzazepin-3-yl)-N-methyl-3-oxo-1-propanamine is in the form of the hydrochloride or fumarate or one of its hydrates or crystalline forms.
